(19) United States
(12) Patent Application Publication
(10) Pub. No.: US 2009/0043210 A1
(43) Pub. Date: Feb. 12, 2009

(54) DATA DETECTION DEVICE AND DATA DETECTION METHOD

(75) Inventors: Shin-ichiroh Kitoh, Tokyo (JP); Po-Chieh Hung, Tokyo (JP)

Correspondence Address: LUCAS & MERCANTI, LLP, 475 PARK AVENUE SOUTH, 15TH FLOOR, NEW YORK, NY, 10016, US

(73) Assignee: KONICA MINOLTA HOLDINGS, INC., Tokyo (JP)

(21) Appl. No.: 12/089,569

(22) PCT Filed: Sep. 26, 2006
(86) PCT No.: PCT/JP2006/319004

§ 371 Date: Apr. 8, 2008

(30) Foreign Application Priority Data
Oct. 12, 2005 (JP) 2005297542

(51) Int. Cl. A61B 6/00

(52) U.S. Cl. 600/476

(57) ABSTRACT

A data detection device (1) includes an illumination unit (3) for illuminating a detection portion of a living body surface so as to obtain a shadow, an image capturing unit (7) for imaging sequential images of the detection portion of the living body surface, and a data processing unit (9) for analyzing the sequential images captured in the image capturing unit (7) so as to analyze the state of shadows, thereby detecting the motion of the living body.

### FIELD OF THE INVENTION

The present invention relates to a data detection device and data detection method, particularly to a data detection device and data detection method for detecting the data on a living body such as a human body.

### BACKGROUND

In the conventional art, a proposal has been made of a device for detecting the data reflecting physiological changes in a living body such as a human body for the purpose of diagnosis such as medical diagnosis. Such detection devices having been proposed so far include a data detection device provided with various forms of detection means.

The Patent Document 1, for example, discloses a blood pressure gauge wherein pressure is applied to a cuff wrapped around a wrist, and hence pressure is applied to the wrist, whereby changes of pressure inside the cuff are detected and blood pressure is measured.

The Patent Document 2 describes a fingerprint image input apparatus wherein the sequential images of the light passing through a finger is captured by a two-dimensional image sensor, and the pulse wave is detected from the temporal change of the transmitted light.

The Patent Document 3 discloses a living body authentication apparatus wherein light is applied to the finger from a light source and the transmitted light is captured as the vein image of the finger in chronological order, whereby the pulsation is detected from changes in the luminance.

The Patent Document 4 discloses a baby incubator wherein the physical condition is extracted and monitored using a video sensor or sound sensor which is not in contact with an infant.

Patent Document 1: Unexamined Japanese Patent Application Publication No. 2002-263073

Patent Document 2: Unexamined Japanese Patent Application Publication No. 2003-144420

Patent Document 3: Unexamined Japanese Patent Application Publication No. 2003-331268

Patent Document 4: Unexamined Japanese Patent Application Publication No. 2004-537335

### DISCLOSURE OF INVENTION

### Problems to be Solved by the Invention

In the blood pressure gauge described in Patent Document 1, a cuff is wrapped around the wrist or the upper arm, and pressure is applied to measure the blood pressure. This requires an apparatus to be fastened onto the human body, and this gives a sense of oppression to the user. Another problem is that the users feel unpleasant when the apparatus is used by a plurality of these users. These problems have been left unsolved.

In the fingerprint image input apparatus described in the Patent Document 2, the fingerprint surface must be kept in contact with the apparatus when the image is captured. Thus, the apparatus of the Patent Document 2 has failed to ensure that the image is captured without being noticed by the user.

In the living body authentication apparatus of the Patent Document 3, the user's finger must be placed at an adequate position in order to ensure accurate authentication. Thus, the user is required to assume a specific posture.

Patent Document 4 fails to describe a specific method for measuring the pulsation and others from the video image.

The object of the present invention is to solve the aforementioned problems and to provide a data detection device and data detection method capable of high precision acquisition of the biological data in non-invasive manner without contact to a living body.

### Means to Solve the Problems

To solve the aforementioned problems, the invention described in Claim 1 provides a data detection device including: an illumination unit for applying illumination light to a detection portion of a living body surface to obtain shadows; an image capturing unit for capturing the sequential images of the detection portion of the living body surface; and a data processing unit for analyzing the sequential images captured by the image capturing unit and the changes in the state of the shadows, thereby detecting the motion of the living body.

According to the invention described in Claim 1, the motion of a living body can be detected in non-invasive manner without contact to the living body by analyzing the sequential images of the detection portion of the living body surface.

The intention described in Claim 2 is the data detection device described in Claim 1 wherein a motion of the living body is pulsation.

According to the invention of Claim 2, pulsation of a subject can be detected as the motion of the living body by analyzing the sequential images.

The invention described in Claim 3 is the data detection device described in Claim 1 or 2 wherein the detection portion of the living body surface is the periphery of the jaws and neck.

According to the invention of Claim 3, high precision detection of the pulsation of a subject is provided by analyzing the sequential images of the periphery of the jaws and neck.

The invention described in Claim 4 is the data detection device described in any one of the aforementioned Claims 1 through 3, characterized by further comprising an illumination position adjusting unit for adjusting the position of the illumination unit to ensure that illumination light is applied obliquely with respect to the front of the living body so that shadows can be easily formed on to the detection portion of the living body surface.

According to the invention of Claim 4, high precision detection of the motion of the living body is achieved through the analysis of the sequential images by capturing a clearer image of the shadow of the living body surface. Further, this invention permits detection to be achieved without the subject being required to assume a specific posture or standing position, because the position of the illumination unit is adjusted and the direction of the illumination light is controlled.

The invention described in Claim 5 is the data detection device described in Claim 4 wherein the illumination unit 3 is composed of the light sources arranged in one- or two-dimensional array, and the aforementioned illumination position adjusting unit controls the direction of the illumination light by switching the position of the light source emitting light in the illumination unit.

According to the invention of Claim 5, the direction of the illumination light can be controlled merely by switching the position of the light source in the illumination unit, without moving the illumination.

The invention described in Claim 6 is the data detection device described in any one of Claims 1 through 5, wherein the illumination unit applies the light of a wavelength band other than visible light to the detection portion of the living body surface.

According to the invention of Claim 6, the illumination unit applies the light of a wavelength band other than visible light. This arrangement permits detection to be achieved without being noticed by the subject.

The invention described in Claim 7 is the data detection device described in any one of Claims 1 through 6, wherein the illumination unit applies near-infrared rays to the detection portion of the living body surface, and the image capturing unit is equipped with an infrared filter that allows passage of the near-infrared rays.

According to the invention of Claim 7, near-infrared rays are applied from the illumination unit. This arrangement provides shadows of high contrast even when a fluorescent lamp is used, because infrared rays are not contained in the fluorescent lamp. Further, shadows of high contrast can be provided because the near-infrared rays are characterized by a high reflectivity on the living body surface.

The invention described in Claim 8 is the data detection method, applying illumination light to a detection portion of a living body surface to be detected so that shadows are formed; capturing sequential images of the detection portion of the living body surface, and analyzing a change in the state of the shadows by analyzing the sequential images, whereby a motion of the living body is detected.

According to the invention of Claim 8, detection of the living body can be achieved in non-invasive manner without contact to the living body, by analyzing the sequential images on the detection portion of the living body surface.

The invention described in Claim 9 is the data detection method described in Claim 8 wherein the motion of the living body is pulsation.

According to the invention of Claim 9, the pulsation of the subject can be detected as the motion of a living body through the analysis of the sequential images.

The invention described in Claim 10 is the data detection method described in Claim 8 or 9, wherein the detection portion of the living body surface is the periphery of the jaws and neck.

According to the invention of Claim 10, high precision detection of the pulsation of a subject is provided by analyzing the sequential images of the periphery of the jaws and neck.

The invention described in Claim 11 is the data detection method described in any one of Claims 8 through 10, characterized by further comprising adjusting the position of the illumination unit to ensure that illumination light is applied obliquely with respect to the front of the living body so that shadows can be easily formed on the detection portion of the living body surface.

According to the invention of Claim 11, high precision detection of the motion of the living body is achieved through the analysis of the sequential images by capturing a clearer image of the shadow of the living body surface. Further, this invention permits detection to be achieved without the subject being required to assume a specific posture or standing position, because the position of the illumination unit is adjusted and the direction of the illumination light is controlled.

The invention described in Claim 12 is the data detection described in Claim 11, wherein the illumination unit composed of the light sources arranged in one- or two-dimensional array is used, and the direction of the illumination light is controlled by switching the position of the light source emitting light in the illumination unit.

According to the invention of Claim 12, the direction of the illumination light can be controlled merely by switching the position of the light source in the illumination unit, without the illumination unit being moved.

The invention described in Claim 13 is the data detection method described in any one of Claims 8 through 12, wherein the light of a wavelength band other than visible light is applied to the detection portion of the living body surface.

According to the invention of Claim 13, the illumination unit applies the light of a wavelength band other than visible light. This arrangement permits detection to be achieved without being noticed by the subject.

The invention described in Claim 14 is the data detection method described in any one of Claims 8 through 13, wherein near-infrared rays are applied to the detection portion of the living body surface, and the sequential images are captured by using an infrared filter that allows passage of the near-infrared rays.

According to the invention of Claim 14, near-infrared rays are applied from the illumination unit. This arrangement provides shadows of high contrast even when a fluorescent lamp is used to illuminate the surrounding area, because infrared rays are not contained in the fluorescent lamp. Further, shadows of high contrast can be provided because the near-infrared rays are characterized by a high reflectivity on the living body surface.

### EFFECTS OF THE INVENTION

The invention of Claim 1 or 8 allows the biological data to be obtained in non-invasive manner without contact to the living body.

The invention of Claim 2 or 9 provides the pulsation data of a subject as the biological data.

The invention of Claim 3 or 10 ensures high precision detection of the pulsation of a subject.

The invention of Claim 4 or 11 provides high precision detection of the motion of a living body, without the subject being required to assume a specific posture or standing position.

The invention of Claim 5 or 12 allows the direction of the illumination light to be controlled merely by switching the position of the light source in the illumination unit.

The invention of Claim 6 or 13 ensures detection to be achieved without being noticed by the subject, whereby data on a living body under normal conditions can be obtained.

The invention of Claim 7 or 14 provides shadows of high contrast.

### BRIEF DESCRIPTION OF THE DRAWINGS

FIG. 1 is a schematic diagram showing part of the data detection device in a first embodiment of the present invention;

FIG. 2 is a chart representing the examples of the emission spectrum of a near-infrared LED and the radiation spectrum of a fluorescent lamp;

FIG. 3 is a chart representing an example of the spectrum of outdoor light;

FIG. 4 is a diagram representing an example of processing in the image capturing unit in a first embodiment of the present invention;

FIG. 5 is a block diagram representing the functional arrangement of the data detection device in a first embodiment of the present invention;

FIG. 6 shows an example of the detection portion of a subject using an image capturing unit in a first embodiment of the present invention;

FIG. 7 is a plan view representing an example of the layout of the illumination unit and image capturing unit in a first embodiment of the present invention;

FIG. 8 is a front view representing another example of the layout of the illumination unit and image capturing unit in a first embodiment of the present invention;

FIG. 9 is a chart showing the average pixel value extracted from the sequential images captured by the image capturing unit in a first embodiment of the present invention;

FIG. 10 is an example of converting into the frequency space the average pixel value extracted from the sequential images; and

FIG. 11 is a plan view representing an example of the layout of the illumination unit and image capturing unit in a first embodiment of the present invention.

### DESCRIPTION OF REFERENCE NUMERALS

1. Data detection device
2. Display unit
3. Illumination unit
3a. LED
4. External device
5. Control unit
6. External communication unit
7. Image capturing unit
8. Memory unit
9. Data processing unit
10. User interface unit
11. Parameter setting/management unit
12. Data accumulation unit
13. Illumination/position adjusting unit
14. I/O unit
15. Rail
20. Network

### BEST MODE FOR CARRYING OUT THE INVENTION

### Embodiment 1

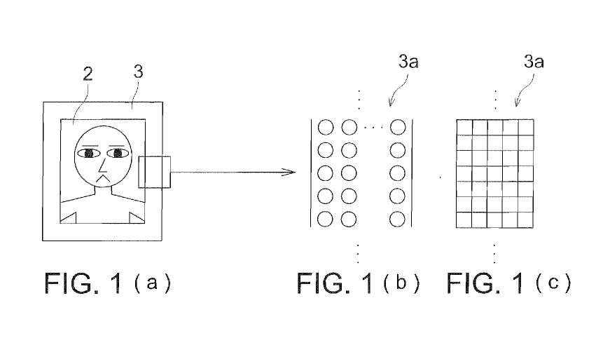

The following describes the first embodiment with reference to FIGS. 1 through 10.

In the data detection device 1 of the present invention, shadows are created on the detection portion of a subject by the illumination light to capture the sequential images and analyze the changes in the state of the shadows in the sequential images, whereby the motion of the living body such as pulsation is detected.

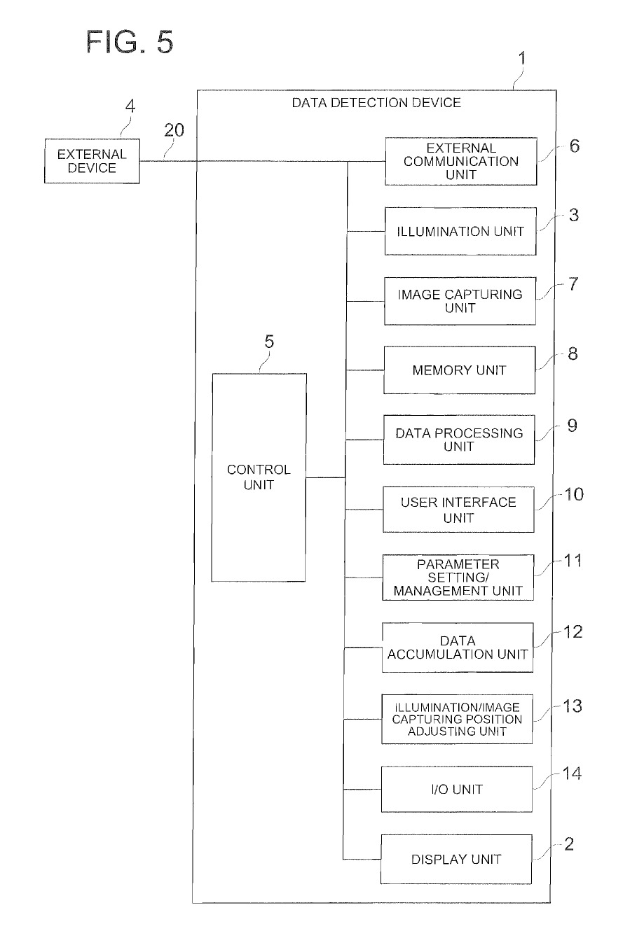

FIG. 1 shows part of the data detection device 1 of the present first embodiment. As shown in FIG. 1 (a), the data detection device 1 of the present embodiment has a display unit 2 which is installed in front of the subject. An image capturing unit 7 (FIG. 5) is installed on the back of the display unit 2 so as to capture the image of a subject. The image capturing unit 7 is mounted on the back of the display unit 2 movably in the lateral or vertical direction so that the direction of imaging the subject can be adjusted.

The display unit 2 can be composed of a CRT, liquid crystal, organic EL, plasma or projection type display, and is so designed that the image data and others obtained by the image capturing unit 7 can be displayed. The display unit 2 of the present embodiment is made up of a half-mirror type material so as to avoid possible problems when an image is captured by the image capturing unit 7.

A illumination unit 3 made up of a plurality of light sources is installed on the edge of the display unit 2, and is designed in such a way that light is applied obliquely with respect to the front of the subject. The partially enlarged view of the illumination unit 3 is shown in FIGS. 1 (b) and (c). The light source 3a of the present embodiment is made up of an LED (Light-Emitting Diode), and a plurality of light sources 3a are arranged in the one- or two-dimensional array. The light source 3a can be a circular light source shown in FIG. 1 (b), or a rectangular light source shown in FIG. 1 (c).

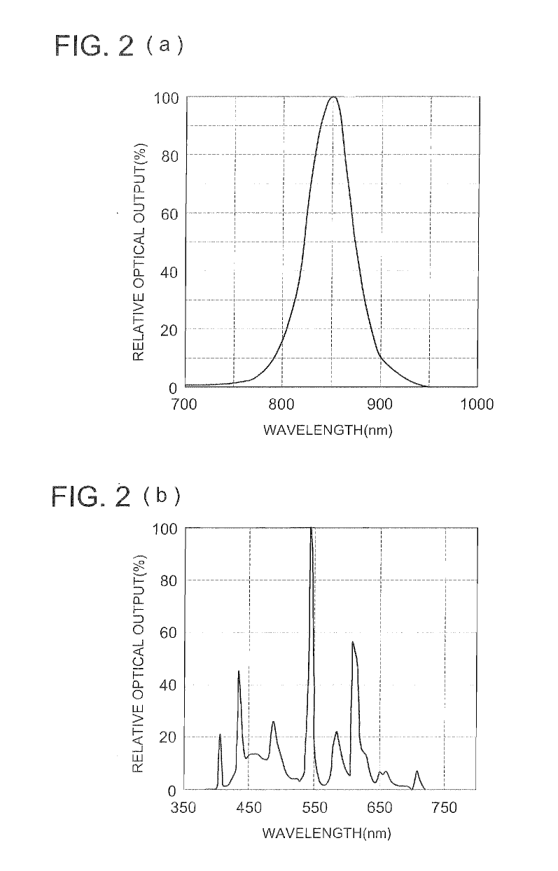

The light source 3a of the illumination unit 3 is preferably similar to a point light source. The present embodiment uses an LED that emits near-infrared light. When the illumination unit 3 emits light of the wavelength band other than visible light, detection can be achieved without being noticed by the subject. FIG. 2 (a) is a chart showing an example of the emission spectrum of the near-infrared LET) as the light source 3a of the present embodiment. The fluorescent lamp used for general room illumination does not radiate the infrared ray having a wavelength of 750 nm or more, as shown in an example of the radiation spectrum of the general fluorescent lamp of FIG. 2 (b). Accordingly, near-infrared light is used for the light source 3a of the illumination unit 3, and an infrared filter is employed in the image capturing unit 7, whereby an image of high contrast can be captured. Further, a fluorescent lamp driven by the general commercial power frequency can be utilized as the light source 3a of the illumination unit 3.

A special-purpose illumination device for illuminating the neck and jaws alone can be separately installed as the illumination unit 3. This special-purpose illumination device can be accommodated in the data detection device 1 in such a way that, when an image is to be captured, an arm is automatically extended and is placed at a predetermined position. Further, it is possible to arrange such a configuration that the position, angle or illumination intensity of this special-purpose illumination device can be controlled.

The illumination on the periphery of the data detection device 1 is only required to be bright enough to avoid possible adverse effect upon creation of the shadow. This illumination is preferred to be as dark as possible. It is preferred that a fluorescent lamp or a white LED that does not emit infrared rays should be used for background illumination and the infrared rays should be emitted from the illumination unit 3 of the data detection device 1 for detection. This arrangement allows the detection to be performed without being noticed by the subject.

When a fluorescent lamp is used for background illumination the illumination light of the illumination unit 3 should be emitted in the reverse phase by synchronization with the fluorescent lamp drive frequency. This arrangement ensures an image to be captured through separation between the image by the background illumination and that by the illumination light of the illumination unit 3. Further, if imaging by the image capturing unit 7 is synchronized with illumination by the illumination unit 3, the image not required for analysis can be removed.

When a white LED is used for background illumination, shadows can be created by using the LED of the wavelength corresponding to the valley thereof since the main wavelength is made up of RGB (red-green-blue). In this case, it is preferred to use an interference filter that allows passage of only the wavelength in the vicinity of that of the illumination light that creates the shadow on the image capturing unit 7.

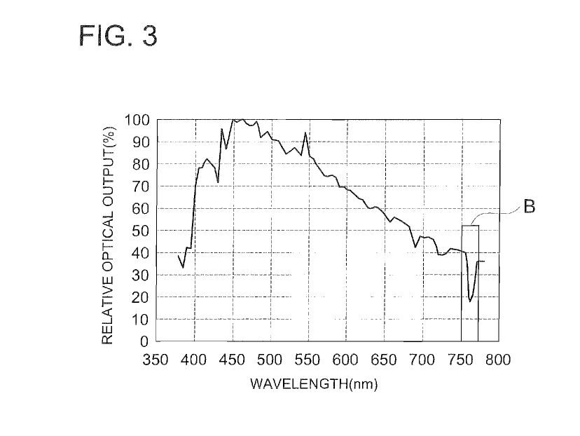

When an image is captured in the illumination environment which tends to be exposed to outdoor light, it is possible to use the light source having a wavelength band characterized by a lower intensity of outdoor light, for example, the light source having the wavelength in wavelength band B, as shown in the chart representing the spectral intensity of the outdoor light in FIG. 3. This does not require alternate lighting between the regular illumination for the subject and the illumination for creating shadows. Further, the interference filter capable of separating between the bands is preferably used in the image capturing unit 7.

The image capturing unit 7 is equipped with the image capturing devices such as a CCD and CMOS, and is made up of one or more than one camera capable of capturing the sequential images of a subject. For example, the image capturing unit 7 can be composed of an auxiliary camera module such as a color or monochromatic video camera, CCD camera, CMOS camera, digital still camera and mobile phone. Further, the image capturing unit 7 is preferably constructed of the camera characterized by a high degree of sensitivity in the near-infrared area and infrared area.

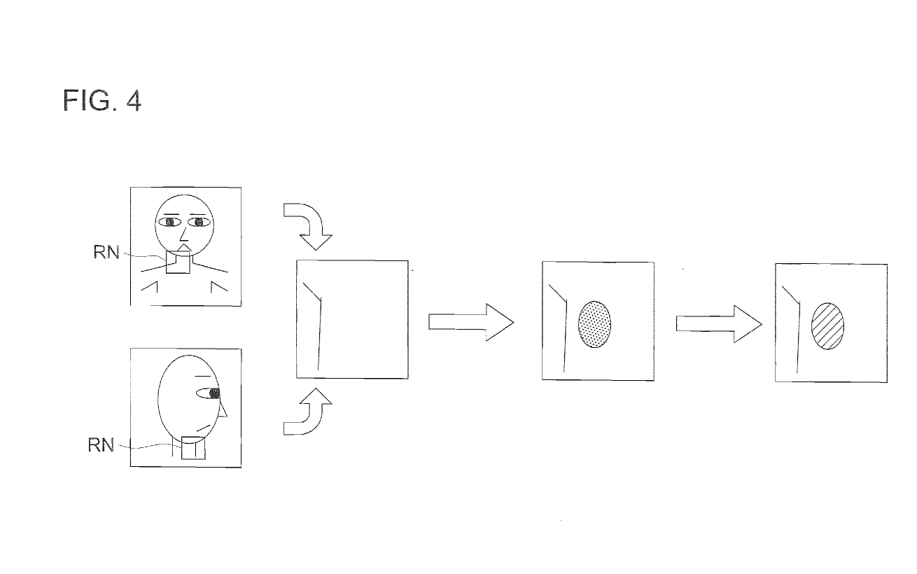

The image capturing unit 7 can be constructed of one camera or a plurality of cameras or camera modules. When one camera is used as the image capturing unit 7, the image of the subject can be captured from the front and the image data on the periphery of the neck can be extracted from the captured image, as shown in FIG. 4. Further, when the image capturing unit 7 is constructed of a plurality of cameras, one of these cameras is used as a special-purpose camera for pulsation detection, and the same processing is applied. When the image capturing unit 7 is constructed of a plurality of camera modules, the module closest to the detection portion of the subject is used as the special-purpose module for pulsation measurement, and the same processing is applied.

For example, the image is captured from the front of the subject, as shown in FIG. 4 (a), or from the right side of the subject, as shown in FIG. 4 (b), and the image on the periphery of the imaging position RN is extracted from the captured image. Based on this data, setting of the imaging position RN is adjusted, as shown in FIG. 4 (c). Then as shown in FIG. 4 (d), the position of the illumination unit 3 is adjusted so that a shadow will be created on the imaging position RN. Lastly, sequential images are captured, as shown in FIG. 4 (e), whereby the image data is obtained.

A special-purpose camera for imaging the neck and jaws can be separately installed as the image capturing unit 7. This special-purpose camera can be incorporated into the data detection device 1 or can be installed at a predetermined position by the arm which is automatically extended at the time of imaging operation. It is also possible to arrange such a configuration that the position, angle, aperture and shutter speed are controlled.

FIG. 5 is a block diagram representing the functional arrangement of the data detection device 1 in the present embodiment. As shown in FIG. 5, the data detection device 1 is connected with an external device 4 via the network 20 so that they can communicate with each other. This arrangement makes it possible send the data on the living body detected by the data detection device 1.

There is no particular restriction to the network 20 of the present embodiment if it permits data communication. The network can be exemplified by the Internet, LAN (Local Area Network), WAN (Wide Area Network), telephone line network, ISDN (Integrated Service Digital Network), CATV (Cable Television) network, and optical communication network. The network for wireless communication as well as wired communication can also be utilized for communication.

The external device 4 is made up of a personal computer and others. It is preferably installed where some sort of consulting or diagnostic services can be provided. Further, the external device 4 can also be constructed as the Internet site wherein consulting information can be obtained, or as a mobile terminal for a consultant, doctor or salesclerk. It is also possible to make such arrangements that, instead of or in addition to the external device 4, a data processing apparatus (not illustrated) is connected to the data detection device 1, wherein this data processing apparatus is capable of analyzing the data such as image data obtained by the data detection device 1 or serving as a database for such data.

As shown in FIG. 5, the data detection device 1 is provided with a control unit 5, external communication unit 6, illumination unit 3, image capturing unit 7, memory unit 8, data processing unit 9, user interface unit 10, parameter setting/management unit 11, data accumulation unit 12, illumination/image capturing position adjusting unit 13, I/O unit 14, and display unit 2. Of these, the illumination/image capturing position adjusting unit 13, I/O unit 14 and display unit 2 are optional components of the data detection device of the present invention.

The control unit 5 is provided with a CPU and RAM so as to control the drive of the components of the data detection device 1. Since the data detection device 1 of the present embodiment handles sequential images, the control unit 5 is preferably made up of chips capable of operation and control at the highest possible speed.

The external communication unit 6 is configured to exchange information with the external device 4 through wired or wireless communication means. Since the data detection device 1 of the present embodiment handles image data, the communication system is preferably designed to ensure transmission at the highest possible speed.

The illumination unit 3 is designed to apply illumination light to create a shadow on the detection portion of the subject at the time of imaging operation. The illumination unit 3 of the present embodiment is constructed to control the direction of emitting the illumination light, by switching the position of the light source for emitting light. However, if there is a big change in the angle of the illumination light during the imaging operation, there will also be a big change in the shadow at that instant. Thus, the data on the captured image must be moved in parallel by the process of correction so that the captured images will be continuous.

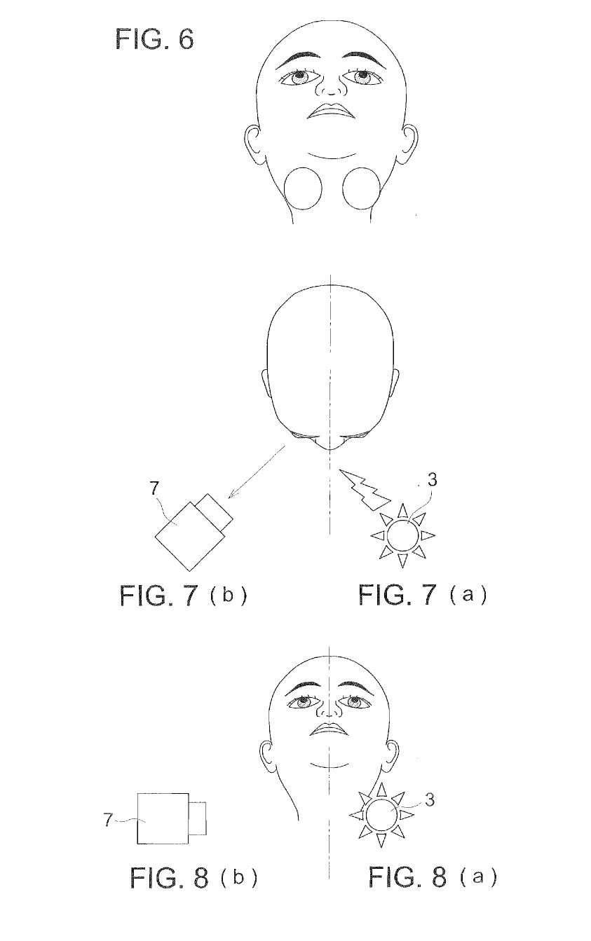

In the present embodiment, since the pulsation closest to the carotid artery causes the motion of the skin surface most conspicuously, the jaws and neck of the subject are determined as the detection portions, as shown in FIG. 6. Thus, illumination light is applied to the jaws and neck of the subject. This makes it possible to observe the changing statuses of the shadow on the portion wherein the scruff of the neck is pulsating in the sequential images.

Thus, to detect the subtle motion on the skin surface close to the carotid artery, the illumination unit 3 is designed to apply illumination light from the direction wherein the shadow can be easily imaged. To be more specific, illumination light is applied obliquely with respect to the front of the subject. For example, the illumination light can be applied about 30 degrees off the front of the subject. It should be noted that this angle is not restricted to 30 degrees, because the optimum angle varies according to the physical size of the subject and the relationship of distance between the illumination unit 3 and image capturing unit 7.

For example, assume that the detection portion of the subject is the right scuff of the neck, as shown in FIG. 7. In this case, the image capturing unit 7 is located in the direction normal to the detection portion (position of FIG. 7 (b)), and the illumination unit 3 is located obliquely to the forward left (position of FIG. 7 (a)). Under this condition, illumination light is applied. This arrangement provides the changing status of the shadow under the most preferred conditions. Further, as shown in FIG. 8, when the detection portion of the subject is determined as the hollow on the side of the Adam's apple, the illumination unit 3 is placed on the same side as the image capturing unit 7 (position of FIG. 8 (b)). This results in direct application of the light, and no shadow is created. Accordingly, illumination light is applied obliquely to the forward left (position of FIG. 8 (a)) or obliquely to the backward left. This arrangement provides the changing status of the shadow under the most preferred conditions. To be more specific, illumination light is applied preferably obliquely to the front of the subject on the side opposite to the detection portion (left side if the detection portion is located to the right of the center of the subject), whatever the direction may be. It should be noted, however, that, if it is tilted excessively to the left, the entire right side will be completely covered with shadow. To avoid this, it is preferably tilted slightly to the left with respect to the front of the subject. The light source of the illumination unit 3 is preferably as high as the Adam's apple.

The image of the lattice or pattern can be formed and projected by the light source of the illumination unit 3. This procedure allows the motion of the living body such as the pulsation to be detected from the distortion of the lattice or pattern.

The illumination unit 3 can be configured in such a way that the position of the light source is shifted synchronously with the vector of the subject motion extracted from the captured image. In this case, means are provided to ensure a constant positional relationship of the light source relative to the detection portion wherein the shadow is created. When the illumination unit 3 is used as a special-purpose device for illumination of the neck and jaws, the arm is moved by the distance corresponding to the compensation for the vector of motion. When the LED as a light source is configured in the one- or two-dimensional array, switching operation is performed to ensure that the position of the light source for emitting light is shifted by that distance.

It is also possible to alternately use the routine illumination of the subject and the illumination for creating shadows. If alternate switching between illuminations (turning on and off of the light) is performed at a high speed beyond human recognition (20 or more cycles per second), a sense of incompatibility is not felt by the subject. The LED is preferred when images are captured by repeating the light on-off operation. Other light sources can also be utilized if the same purpose can be fulfilled.

If one and the same data detection device 1 is used to examine a plurality of subjects, the optimum illumination angle differs according to each subject. Accordingly, this is preferably stored in the parameter setting/management unit 11, and the position of the light source is preferably switched using the method of manual input or facial authentication in the subject interface unit 10. In this case, to find out the optimum illumination, angle for each subject, the subject is placed at an adequate position, and is requested to put his hand to the portion wherein the most conspicuous pulsation is left by the subject. This position can be used for detection.

The image capturing unit 7 serves the function of image capturing means. The detection portion of the subject wherein shadows are created by the illumination light of the illumination unit 3 is imaged and formed into sequential images. The sequential images are used to observe the changing status of the shadow.

As shown in FIG. 7, for example, when the right scruff of the neck is determined as the detection portion of the subject, the image capturing unit 7 can be installed obliquely to the forward right with respect to the front of the subject (position of FIG. 7 (b)) so that the right scruff will be located on the right. The image capturing unit 7 can be placed about 30 degrees off the front of the subject. It should be noted that this angle is not restricted to 30 degrees, because the optimum angle varies according to the physical size of the subject and the relationship of distance between the illumination unit 3 and image capturing unit 7. Further, the left scruff can be selected as the detection portion. Furthermore, the image capturing unit 7 can be installed on the front of the subject or obliquely to the forward left. The image can also be captured from the bottom by the operator who is looking at the subject from below.

As shown in FIG. 8, when the detection portion of the subject is determined as the hollow on the side of the Adam's apple, the image capturing unit 7 can be placed so that the image is captured in one direction of the right (position of FIG. 8 (b)) or left, in such a way that the side of the Adam's apple will be located at the front position. The image capturing unit 7 is preferably placed as high as the Adam's apple of the subject.

The subject can face the front when imaged by the image capturing unit 7. A clearer shadow can be captured if the subject faces slightly upward as if he were rinsing his mouth, as shown in FIG. 6 or 8. For example, the direction wherein the subject should face is marked, or the on-off operation of the light source is performed so as to call the attention of the subject. This allows the imaging operation to be made with the face of the subject turned upward.

It is also possible to make such arrangements that the display unit 2 of FIG. 1 is made up of an electronic display so that the instruction on the position of the face is displayed according to the information of the stereoscopic camera installed separately from the detection camera. Instead of the display unit 2, a mirror can be used. When a mirror can be used in place of the display unit 2, a mark is put at the center of the mirror or on the mirror per se, and the face is placed on top of this mark, whereby the position of the subject is determined. Further, the mirror or display is provided with a position adjusting function, in such a way that the position is automatically adjusted to keep the position of the subject face aligned with the mark.

To get accurate data by controlling the movement of the subject, some indication is preferably given close to the image capturing unit 7 during the imaging operation. For example, in addition to the aforementioned mark or light on-off operation, the display pattern or color can be changed in the display unit 2, or an animation can be shown if the subject is a child.

The image capturing unit 7 captures the sequential images of the detection portion of the subject for at least two seconds. When the imaging time is two seconds or more as in this example, the sequential images corresponding to two cycles of pulsation can be provided. As the imaging time is longer, more accurate detection of pulsations is obtained. However, this also means that the load given to the subject is increased accordingly.

When a fluorescent lamp is used as the light source of the illumination unit 3, the image capturing unit 7 must be provided with a mechanism of reducing or suppressing the flicker function. Further, the adjusting functions for adjusting the aperture of the image capturing unit 7, the shutter speed and the number of the frames of the sequential images can be preferably set automatically or manually. There is no particular restriction to the number of the frames of the sequential images if the motion of the subject can be reproduced smoothly.

It is also possible to make such arrangements that the image of the face of the subject located at a predetermined position during the imaging operation is captured by the stereoscopic camera installed separately from the detection camera in the image capturing unit 7, and the posture of the subject is detected from that captured image, thereby determining the position of the illumination unit 3 and image capturing unit 7. Further, a stereoscopic camera can be formed by the detection camera and another monocular camera. The movement of the subject is constantly monitored by this stereoscopic camera. Thus, an alarm is preferably displayed when there is an excessive approach or separation of the subject, or there is a change in the angle with respect to the illumination unit 3. When adequate conditions (position and angle) have been met, indication to that effect can be given.

To avoid the possible fluctuation in the pulsation of the subject conscious of the pulsation being examined, quiet music or aroma can be produced in such a way that the subject will be relaxed at the time of imaging operation of the image capturing unit 7.

The memory unit 8 is made up of the RAM, ROM, DIMM and others. The data required in the data processing unit 9 and others is transferred from the data accumulation unit 12 to the memory unit 8, where the data is stored temporarily. This arrangement ensures high-speed and steady operations of the data detection device 1. Further, the memory unit 8 of the present embodiment is required to have the storage capacity to permit processing of the sequential images on a real time basis without missing any frame.

The data processing unit 9 detects the motion of the living body such as pulsation by analyzing the changing status of the shadow in the sequential images captured by the image capturing unit 7.

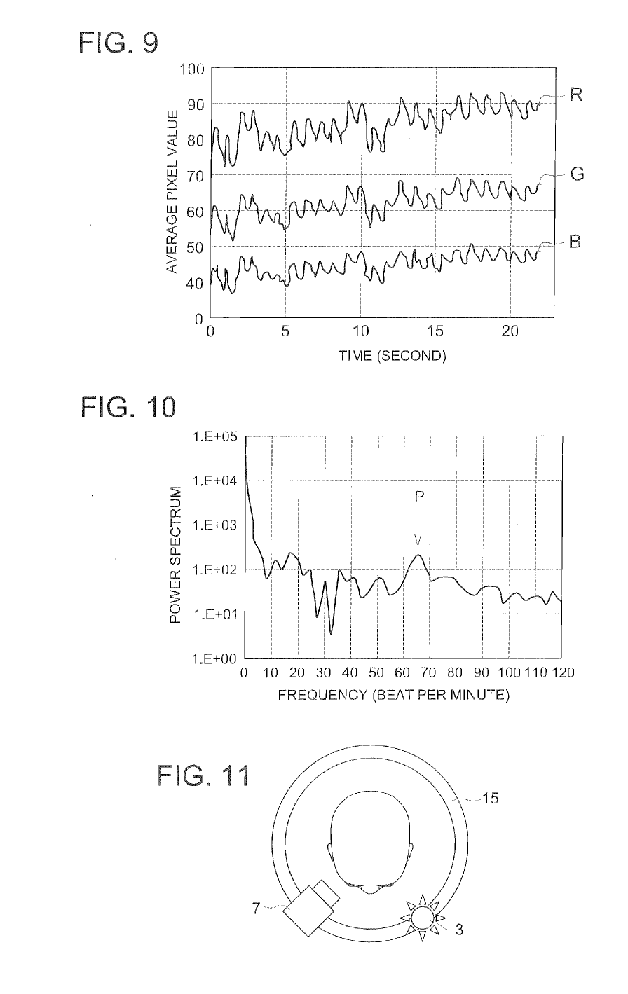

To be more specific, the data processing unit 9 of the present embodiment calculates the average pixel value of the shadow in the detection portion for each frame of the sequential images, and accumulates the average pixel values after each passage of imaging time, as shown in FIG. 9. This procedure allows the state of the pulsation of the subject to be observed. FIG. 9 shows the average pixel values for each imaging time in the color sequential images. For example, it shows the average pixels values of the red (R), green (G) and blue (B) as counted from the top. One graph is used for near-infrared imaging operations.

The “shadow” of the detection portion can be the overall shadow in the captured image, or a predetermined rectangular region specified from the shadow of the captured image. In this case, the average pixel value is calculated from the relationship between the area of the rectangular region and the average particle value. Further, the (average) pixel value can be calculated from one pixel of the shadow in the captured image.

It is also possible to make such arrangements that the vector of the subject motion is extracted from the motion of the shadow portion of the captured image and other texture (outline of the scruff of the neck and jaws), thereby moving the region of the pixel to be averaged, with consideration given to the motion vector.

For a predetermined position in the captured image (or the average value between the predetermined position and the position surrounding that predetermined position, it is also possible to carry out the procedures wherein changes of the sequential images in chronological order are all put to Fourier transformation, information on the position indicating the change in the frequency which appears to represent the most conspicuous pulsation is stored, and the pixels of the periphery are averaged.

The data processing unit 9 can detect the number of the pulsations of the subject by counting the peaks (or valleys) per minute in the chart representing the changes of average pixel values in chronological order.

Further, other feature volumes can be extracted from the chart showing the changes of the average pixel values in chronological order as shown in FIG. 9. For example, the degree of unequal spacing of the pulsation (irregular heartbeats) can be detected from the difference in the peak spacing in the chart. Blood pressure can be estimated from the average pixel values in the peak of the chart.

In the analysis of the sequential images, when the sequential images include the portion wherein the pixel value is very low, the scruff of the subject may be covered with hair or beard, which will affect the result of detection. To avoid this possibility, such a message as “Remove your hear.” or “Put your hair together at the back.” can be displayed on the display unit 2 to alert the operator. If the motion of the detection portion cannot be clearly identified by the beard, such a message as “Remove your beard by shaving” can be displayed to warn the subject.

In the data processing unit 9, the chart representing the changes of the average pixel values in chronological order shown in FIG. 9 can be transformed into the frequency space as shown in FIG. 10 and the pulses can be counted. Fourier transformation (or wavelet transformation) is used in this case. Then the peak values other than the DC component of the power spectrum are correlated with the pulse rate. The health conditions can be estimated from the percentage of the presence of the frequency components. This procedure is effectively used especially when the sequential image data contains noise.

When the detection portion is moved by other than pulsation, —for example, when the subject swallows his saliva—, the data processing unit 9 allows the changes of the average pixel values in chronological order to be transformed into the frequency space by Fourier transformation, and separates them from the low frequency component, whereby the pulse rate is detected. To be more specific, when the detection portion is moved by the factor other than the pulsation, the influence of low frequency is given to the average pixel values. As shown in FIG. 10, changes in the average pixel values in chronological order are transformed into the frequency space by Fourier transformation and are separated from the low frequency components. The frequency P which reaches the peak with the high frequency component is detected from the lower limit in the pulse rate that can be measured, whereby the pulse rate data can be obtained.

The user interface unit 10 is made up of a keyboard, mouse, track ball and others. It allows the user's instruction to be inputted, and permits the current status of the data detection device 1 and the request thereof to be conveyed to the user. The conventional interface such as the keyboard, mouse, track ball and others can be utilized, but the apparatus is preferably configured to minimize the user's load. Thus, it can be integrated with the display unit 2 to form a touch panel, which constitutes the interface. Further, it is preferred to configure a system by installing an acoustic apparatus such as a speaker and microphone in such a way that communication is provided by the voice, gesticulation or gesture of the user (including a sophisticated communication means such as a sign language).

The parameter setting/management unit 11 is designed to set the parameters on the control of various components of the data detection device 1 such as control of imaging by the image capturing unit 7 and control of data processing by the data processing unit 9, and to manage the parameters having been set.

The data accumulation unit 12 is designed to manage and store the image data inputted from the outside, the image data having been processed by the data detection device 1 or the temporary data in the middle of image processing.

The illumination/image capturing position adjusting unit 13 automatically adjusts the positions of the illumination unit 3 and image capturing unit 7 for the purpose of capturing desired sequential images. It is also possible to perform manual adjustment by inputting instructions through the user interface unit 10.

The external device 14 can be connected with a bimetal sensor as a means for acquiring the biological data (e.g., thermometer, weighing machine, body fat ratio scale, blood pressure gauge, electrocardiograph, skin age gauge, bone density gauge, and pulmometer), and with the equipment for handling portable devices such as various forms of memory cards. Various forms of data required for setting the operation of the data detection device 1 can be inputted or outputted from such equipment.

In addition to the image data captured by the image capturing unit 7, the image data being processed by the data processing unit 9 and the image data stored in the data accumulation unit 12, the display unit 2 displays information on the status of the components of the data detection device 1 and information sent from the external device 4.

The following describes the data detection method of present invention using the aforementioned data detection device 1.

When the subject has come closer to the data detection device 1, the illumination/image capturing position adjusting unit 13 adjusts the position to ensure that the image capturing unit 7 can easily image the detection portion of the subject. It is also possible to carry out imaging operations by installing the image capturing unit 7 at predetermined position, without using the illumination/image capturing position adjusting unit 13.

In the present embodiment, the jaws and neck of the subject are determined as the detection portion, as shown in FIG. 6. The illumination/image capturing position adjusting unit 13 adjusts the image capturing unit 7 so that it will be the positioned as indicated in FIG. 7 (b) or FIG. 8 (b). As described above, the image capturing unit 7 can be installed obliquely to the forward right or left of the subject, as well as on the front of the subject or at the position wherein the operator looks at the subject from below. The image capturing unit 7 is preferably placed as high as the Adam's apple of the subject.

In this case, it is also possible to carry out the procedures wherein the face of the subject is imaged by a stereoscopic camera installed separately from the detection camera of the image capturing unit 7, and the posture of the subject is detected from this captured image, whereby the position of the image capturing unit 7 is determined.

To adjust the direction of the subject relative to the image capturing unit 7 and to get a clearer shadow, a mark can be put in the direction wherein the subject should face, or the on-off operation of the light source can be performed so as to call the attention of the subject and to have his face turned upward.

Further, the display unit 2 of FIG. 1 can be used as an electronic display, and the face position can be specified and displayed according to the information of the stereoscopic camera installed separately from the detection camera. Furthermore, instead of the display unit 2, a mirror can be used. When a mirror can be used in place of the display unit 2, a mark is put at the center of the mirror or on the mirror per se, and the face is placed on top of this mark, whereby the subject can be positioned. Further, the mirror or display is provided with a position adjusting function, in such a way that the position is automatically adjusted to keep the position of the subject face aligned with the mark.

The illumination/image capturing position adjusting unit 13 switches the position of the light source for emitting light in the illumination unit 3 to ensure that illumination light is applied in the direction wherein the shadow of the detection portion can be easily captured. To be more specific, as shown in FIG. 7 (a) or FIG. 8 (a), the position of the light source is adjusted so that light will be applied obliquely to the front of the subject. Further, the illumination unit 3 is preferably placed as high as the Adam's apple of the subject.

Further, the illumination unit 3 can be configured in such a way that the position of the light source is shifted synchronously with the vector of the subject motion extracted by the data processing unit 9 from the image captured by the image capturing unit 7. In this case, means are provided to ensure a constant positional relationship of the light source relative to the detection portion wherein the shadow is created.

If one and the same data detection device 1 is used to examine a plurality of subjects, the illumination light angle best suited to each user can be read from the parameter setting/management unit 11, and the position of the light source can be switched using the method of manual input or facial authentication in the subject interface unit 10.

To avoid the possible fluctuation in the pulsation of the subject conscious of the pulsation being examined, quiet music or aroma can be produced in such a way that the subject will be relaxed during the imaging operation.

When the positions of the image capturing unit 7 and illumination unit 3 relative to the subject have been determined in this manner, the illumination unit 3 applies illumination light to the detection portion and to create a shadow.

In this case, it is also possible to use the method of alternate lighting between the regular illumination for the subject and the illumination for creating shadows. In this case, alternate switching between illuminations is performed at a speed of 20 or more cycles per second.

The image of the lattice or pattern can be formed and projected by the light source of the illumination unit 3. This procedure allows the motion of the living body such as the pulsation to be detected from the distortion of the lattice or pattern.

When a fluorescent lamp is used for background illumination, the illumination light of the illumination unit 3 should be emitted in the reverse phase by synchronization with the fluorescent lamp drive frequency, and the illumination should be synchronously with the imaging operation by the image capturing unit 7.

Then the image capturing unit 7 captures the sequential images of the detection portion wherein a shadow is formed by the illumination light of the illumination unit 3.

When one camera is used as the image capturing unit 7, the image of the subject can be captured from the front and the image data on the periphery of the neck can be extracted from the captured image, as shown in FIG. 4. Further, when the image capturing unit 7 is constructed of a plurality of cameras, one of these cameras is used as a special-purpose camera for pulsation detection. When the image capturing unit 7 is constructed of a plurality of camera modules, the module closest to the detection portion of the subject is used as the special-purpose module for pulsation measurement.

The image capturing unit 7 captures the sequential images of the detection portion of the subject for at least two seconds. This procedure provides the sequential images corresponding to two cycles of pulsation.

To get accurate data by controlling the movement of the subject, some indication is preferably given close to the image capturing unit 7. For example, in addition to the aforementioned mark or light on-off operation, the display pattern or color can be changed in the display unit 2, or an animation can be shown.

The data processing unit 9 detects the motion of the living body such as pulsation by analyzing the changing status of the shadow in the sequential images captured by the image capturing unit 7.

To be more specific, the data processing unit 9 calculates the average pixel value of the shadow in the detection portion for each frame of the sequential images, and accumulates the average pixel values after each passage of imaging time, as shown in FIG. 9. This procedure makes it possible to observe the state of the pulsation of the subject including the pulse rate and degree of unequal spacing of the pulsation (irregular heartbeats).

In the analysis of the sequential images, when the sequential images include the portion wherein the pixel value is very low, and it has been determined that the scruff of the subject is covered with hair or beard, a message can be displayed on the display unit 2 to alert the operator.

In the data processing unit 9, the chart representing the changes of the average pixel values in chronological order shown in FIG. 9 can be transformed into the frequency space as shown in FIG. 10 and the pulses can be counted. When the detection portion is moved by other than pulsation, the data processing unit 9 allows the changes of the average pixel values to be transformed into the frequency space by Fourier transformation, and separates them from the low frequency component, whereby the pulse rate is detected.

As described above, according to the data detection device and data detection method of the present invention, the motion of a living body can be detected in non-invasive manner without contact to the living body by analysis of the sequential images of the detection portion on the body surface.

The pulsation of the subject can be detected as the motion of a living body.

High-precision detection of the subject pulsation can be provided by the analysis of the sequential images of the jaws and neck.

By capturing a clearer image of the shadow of the living body surface, high-precision detection of the motion of the living body can be provided by the analysis of the sequential images. Further, the position of the illumination unit 3 is adjusted and the direction of the illumination light is controlled. This arrangement permits detection to be performed without having to request the subject to assume a specific posture or standing position.

Since the illumination unit 3 made up of the light sources 3a installed in one- or two-dimensional array is employed, the direction of the illumination light can be controlled merely by switching the position of the light source 3a in the illumination unit 3, without the need of moving the illumination unit 3.

The light of a wavelength band other than visible light is emitted from the illumination unit 3. This arrangement ensures the detection to be performed without being noticed by the subject.

Since near-infrared light is emitted from the illumination unit 3, a shadow of high contrast can be obtained even when a fluorescent lamp is used for background illumination. This is because the fluorescent lamp does not include infrared radiation. Further, the near-infrared light is characterized by a high reflectivity on the surface of a living body, and this characteristic provides a shadow characterized by high contrast.

### Embodiment 2

The following describes the second embodiment of the present invention with reference to FIG. 11. The same components as those of the first embodiment will be assigned with the same numerals of reference, and will not be described to avoid duplication.

As shown in FIG. 11, the data detection device 1 of the present embodiment is provided with a circular or elliptical rail 15 which is to be laid around the subject. The rail 15 can be either curved as part of a circle or ellipse, or linear. In the present embodiment, the subject stands or sits down inside the rail 15.

The illumination unit 3 and image capturing unit 7 of the present embodiment are installed movably on the rail 15, and are designed to permit free adjustment of the angle in the direction of emitting the illumination light to the subject or the direction of image capturing.

The user interface unit 10 is so constructed as to allow the instruction on the detection portion of the subject to be inputted. In the present embodiment, the neck of the subject is imaged as the default of the detection portion. If the user interface unit 10 is used to specify another portion (e.g., wrist, ankle or temples), that portion is imaged.

The illumination/image capturing position adjusting unit 13 is an essential component of the data detection device 1 in the present embodiment. The illumination/image capturing position adjusting unit 13 moves the illumination unit 3 and image capturing unit 7 in the rail 15 according to the input instruction from the user interface unit 10 and determines the position.

The position of the image capturing unit 7 can be adjusted by matching between the captured image and the information of the template and table corresponding to the detection portion while moving the image capturing unit 7. For example, if the detection portion is the “right scruff”, the table holds the information of “right scruff=“scruff” on the “right side” of the “the Adam's apple” of the “neck”, and the position can be adjusted by using the template of the “neck” or “the Adam's apple” placed under the management of the parameter setting/management unit 11, and the feature amount thereof.

In the step of adjusting the position of the illumination unit 3, coarse adjustment of made using the area or density (the degree of smallness in the pixel value) of the shadow formed on the detection portion. This is followed by the step of making a fine adjustment using the maximum point or minimum point as the center. In the process of fine adjustment, a step is taken to detect the change of shadow in chronological order, and to find out the position wherein the peak and valley of changes in the pixel value can be detected most effectively.

In the present embodiment, the illumination unit 3 and image capturing unit 7 can be moved on the rail 15. However, it is also possible to arrange such a configuration that the height of the illumination unit 3 and image capturing unit 7 can be adjusted, wherever required.

The illumination/image capturing position adjusting unit 13 is designed in such a way that only the position of the illumination unit 3 and image capturing unit 7 can be adjusted. However, it is also possible to adopt such a structure wherein the shadow of the detection portion is adjusted by controlling the camera parameter such as the aperture and shutter speed in the image capturing unit 7 or the intensity of illumination in the illumination unit 3. Further, it is also possible to adopt such a structure wherein the recursively optimum positions of the illumination unit 3 and image capturing unit 7 is determined by repeating the adjustment of the illumination unit 3 and image capturing unit 7 wherever required. In this case, if the rail 15 is linear, the camera parameter of the image capturing unit 7 and the intensity of the illumination of the illumination unit 3 are controlled according to the position on the rail.

The following describes the data detection method of the present invention using the aforementioned data detection device 1:

When the subject has come closer to the data detection device 1, the illumination/image capturing position adjusting unit 13 moves the image capturing unit 7 on the rail 15 according to the instruction inputted from the user interface unit 10, and automatically adjusts the position so that the detection portion of the subject can be easily imaged.

In this case, the position of the image capturing unit 7 is adjusted by matching between the captured image and the information of the template and table corresponding to the detection portion while moving the image capturing unit 7.

The illumination/image capturing position adjusting unit 13 moves the illumination unit 3 on the rail 15, and automatically adjusts the position wherein illumination light is applied in the direction wherein the shadow of the detection portion can be easily captured.

In this case, coarse adjustment is made using the area or density (the degree of smallness in the pixel value) of the shadow formed on the detection portion. This is followed by the step of making a fine adjustment using the maximum point or minimum point as the center. In the process of fine adjustment, a step is taken to detect the change of shadow in chronological order, and to find out the position wherein the peak and valley of changes in the pixel value can be detected most effectively.

When the illumination unit 3 and image capturing unit 7 have been positioned in the aforementioned manner, the illumination unit 3 applies illumination light to the detection portion of the subject to form a shadow on the detection portion, whereby the image capturing unit 7 captures the image of the detection portion. In the step of the image capturing operation, the image capturing unit 7 images the neck of the subject as the default of the detection portion. If the user has inputted the name of the detection portion of the subject using the user interface unit 10, that portion is imaged.

As described above, according to the data detection device and data detection method of the present invention, the illumination unit 3 and image capturing unit 7 can be positioned easily by moving the illumination unit 3 and image capturing unit 7 on the rail 15.

The position of the image capturing unit 7 is adjusted by matching between the captured image and the information of the template and table corresponding to the detection portion while moving the image capturing unit 7. This procedure ensures high-precision positioning of the image capturing unit 7.

As described above, according to the data detection device and data detection method of the present invention, biological data can be obtained in non-invasive manner without contact to the living body.

The pulsation data of a subject can be obtained as the biological data.

High-precision detection of the pulsation of the subject is provided.

High-precision detection of the motion of the living body is ensured. Detection can be performed without having to request a subject to assume a special posture or standing position.

Since the illumination unit is made up of the light sources arranged in one- or two-dimensional array, the direction of the illumination light can be controlled merely by switching the position of the light sources in the illumination unit.

The biological data under normal conditions can be obtained since detection is performed without being noticed by the subject.

Further, the shadow of high contrast can be obtained.

1. A data detection device comprising:
an illumination unit for applying illumination light to a detection portion of a living body surface to obtain shadows;
an image capturing unit for capturing sequential images of the detection portion of the living body surface; and
a data processing unit for analyzing the sequential images captured by the image capturing unit and the changes in the state of the shadows, thereby detecting a motion of the living body.

2. The data detection device described in claim 1 wherein the motion of the living body is pulsation.

3. The data detection device described in claim 1, wherein the detection portion of the living body surface is the periphery of the jaws and neck.

4. The data detection device described in claim 1, characterized by further comprising an illumination position adjusting unit for adjusting the position of the illumination unit to ensure that illumination light is applied obliquely with respect to the front of the living body so that shadows can be easily formed on to the detection portion of the living body surface.

5. The data detection device described in claim 4 wherein the illumination unit is composed of the light sources arranged in one- or two-dimensional array, and the illumination position adjusting unit controls the direction of the illumination light by switching the position of the light source emitting light in the illumination unit.

6. The data detection device described in claim 1, wherein the illumination unit applies the light of a wavelength band other than visible light to the detection portion of the living body surface.

7. The data detection device described in claim 1, wherein the illumination unit applies near-infrared rays to the detection portion of the living body surface, and the image capturing unit is equipped with an infrared filter that allows passage of the near-infrared rays.

8. A data detection method comprising;
applying illumination light to a detection portion of a living body surface to be detected so that shadows are formed;
capturing sequential images of the detection portion of the living body surface; and
analyzing a change in the state of the shadows by analyzing the sequential images, whereby a motion of the living body is detected.

9. The data detection method described in claim 8 wherein the motion of the living body refers to pulsation.

10. The data detection method described in claim 8, wherein the detection portion of the living body surface refers to the periphery of the jaws and neck.

11. The data detection method described in claim 8, characterized by further comprising adjusting the position of the illumination unit to ensure that illumination light is applied obliquely with respect to the front of the living body so that shadows can be easily formed on the detection portion of the living body surface.

12. The data detection method described in claim 11, wherein the illumination unit composed of the light sources arranged in one- or two-dimensional array is used, and the direction of the illumination light is controlled by
